(19) United States
(12) Patent Application Publication
(10) Pub. No.: US 2002/0072676 A1
(43) Pub. Date: Jun. 13, 2002

(54) Apparatus and method ofor spectroscopic analysis of human or animal tissue in or body fluids

(76) Inventor: Natalia I. Afanassieva, Reno, NV (US)

Correspondence Address: Robert C. Hall, Sierra Patent Group, Ltd., P.O. Box 6149, Stateline, NV, 89449, US

(21) Appl. No.: 09/783,266

(22) Filed: Dec. 1, 2000

Related U.S. Application Data
(63) Continuation of application No. 09/172,186, filed on Oct. 13, 1998, now abandoned.
(60) Provisional application No. 60/063,054, filed on Oct. 27, 1997.

(51) Int. Cl. A61B 5/00

(52) U.S. Cl. 600/473

(57) ABSTRACT

The present invention relates to methods employing fiberoptic evanescent wave Fourier transform infrared (FEW-FTIR) spectroscopy using fiberoptic sensors operated in the attenuated total reflection (ATR) regime in the middle infrared (MIR) region of the spectrum (850 to 4000 cm⁻¹). The apparatus and method claimed is applied to diagnostics and characterization of noninvasive and rapid (seconds) direct measurements of spectra (in real time) of normal and pathological tissues in vivo, ex vivo and in vitro. The aim of our invention is testing and monitoring of normal skin and various skin tumor tissues at the early stages of their development. Furthermore the apparatus and method is suitable for fluid diagnostics, as well as endoscopic and biopsy applications. Specifically the remote diagnostics of normal skin and malignant tissue on the skin surface (directly on patient) can distinguish between normal and malignant skin. In addition the apparatus and method can be applied for different types of clinical diagnostics. Finally the invention relates to diagnostics of environmental damage of skin tissue and acupuncture points, and treatment of skin tissue on a molecular level.

### BACKGROUND PRIOR ART

Fourier transform infrared (FTIR) spectroscopy monitoring techniques have been discussed, for example in Bornstein et al. U.S. Pat. No. 5,070,243, and Bornstein and Lowry U.S. Pat. No. 5,436,454. In the U.S. Pat. No. 5,070,243 Bornstein et al. claim unclad optical waveguides as probes for fluid medium to increase the sensitivity of spectroscopic measurements by the ATR method. However, the sensors and waveguides claimed are not suitable for tissue diagnostics in vivo. In the U.S. Pat. No. 5,436,454 (1995) Bornstein and Lowry describe another optical probe for remote attenuated total reflectance measurements of liquid, and/or relatively solid materials. Their fiber probes are quite rigid and are characterized by a waveguide element in the form of a loop. In addition, chalcogenide glass is used as the fiber material. These suggested probes are not very practicable for nontoxic, noninvasive tissue diagnostics in vivo. Furthermore the epoxy used for material sealing and the chalcogenide glass as a fiber probe may be toxic and therefore not suitable for tissue diagnostics in vivo. Stevenson et al., in U.S. Pat. No. 5,585,634 (1996) claims attenuated total reflectance sensing with U shaped probes consisting of optical fibers with core cladding, where only the U shaped sensor surface portion is uncladded. This method is limited by the selection of fiber material (chalcogenide glass) and the complex shape of the fiber probe, and requires extended sensing time. In addition, Stevenson does not claim any tissue applications in vivo.

Weissman et al. , U.S. Pat. No. 5,569,923 discloses a fiber optic reflectance probe for the FTIR and ATR regime. The probe is made of chalcogenide glass and has not been optimized for tissue diagnostics in vivo. Devices and methods for optical and spectroscopic methods for tissue diagnostics or analysis of biological materials are described in U.S. Pat. Nos. 5,280,788, and 5,349,954. In particular the invention of James et al. U.S. Pat. No. 5,280,788 relates to optical spectroscopy in the diagnosis of tissue where a needle probe is in close contact with the tissue surface. However this method utilizes dye lasers as a light source and is therefore not very convenient for clinical applications. The U.S. Pat. No. 5,349,954 by Tiemann et al. proposes an instrument for characterizing tumor tissue, specifically mammographically abnormal tissue, with a broad band light source and monochromator. This cancer diagnostic technique uses a hollow needle, fiber optic illuminator for breast tissue detection. This method can only analyze shifts in hemoglobin oxygenation. Evans suggests in U.S. Pat. No. 5,419,321 a non-invasive medical sensor for living tissue such as skin tissue or organs, where the noninvasive monitoring process is not specified in detail. This patent is based on the non-invasive determination of analyte concentration in the bodies of mammals, in particular the concentration of glucose in blood. Stoddart and Lewis in U.S. Pat. No. 5,349,961 disclose a methodology and apparatus for the clinical evaluation of biological matter, related to internal tissue characterization of skin pigmentation, on a nonintrusive in vivo basis. The examination and/or analysis of tissue and/or biological materials is performed by optical spectrometry in the visible and near infrared range, which do not provide molecular vibrational band information.

### BACKGROUND OF THE INVENTION

This invention is concerned with a new combination of Fourier Transform Infrared FTIR) Spectroscopy and fiber optics technology in the middle infrared region from about 3 to 20 microns. Furthermore this invention relates to the diagnostics of normal and pathological tissues in vivo. In particular nontoxic, chemically inert, nonhygroscopic, intrinsically safe, flexible, low loss optical fiber probes are used for noninvasive or minimally invasive, fast, direct, remote measurements of infrared spectra from tissue in vivo.

The present invention relates to a new complex spectroscopic method and applications using middle infrared optical fiber probes for noninvasive diagnostics of normal, precancerous, and cancerous human tissue in vivo as well as other biological tissues and/or fluids at a molecular level.

The present invention elucidates new trends and methods of noninvasive diagnostics of biotissues in vivo, where more advanced technologies are combined including fiberoptic evanescent wave Fourier transform infrared (FEW-FTIR) spectroscopy tools using extremely low loss fibers with different configurations of fiber optical probes and sensors operated in the ATR regime in the middle infrared (MIR) wavelength range (800 to 4000 cm⁻¹). In particular these methods have the following unique properties: nondestructive, noninvasive, nontoxic, chemically inert, intrinsically safe, nonhygroscopic, fast (seconds), direct, remote, realtime, in vivo, ex vivo and in vitro tissue diagnosis. These techniques are simple and are characterized by low-cost maintenance and are therefore suitable to any commercial application of FEW-FTIR spectrometer including clinical applications.

In particular the potential of the method of this invention is huge for characterizing normal and pathological tissue of the human or animal body (see FIGS. 1 and 2). Hence this combination of fiber optical sensors and FT spectrometers can be applied to many fields: (i) noninvasive medical diagnostics of cancer and other disease states in vivo, (ii) monitoring of biochemical processes, (iii) surface diagnostics of numerous materials, (iv) minimally invasive bulk diagnostics of tissues and materials, (v) characterization of the quality of food, pharmacological products and cosmetics (vi) characterization and treatment of aging of the skin, etc.

This invention is concerned with bare-core (unclad) fibers used in different configurations of probes in the ATR regime of FTIR spectroscopy for spectroscopic monitoring and diagnostics in real time of skin tissue in vivo, ex vivo and in incisions (see FIG. 6). The invention includes also nontoxic, minimally invasive, remote, fast, and ex vivo characterization of normal and abnormal tissue from breast, stomach, lung, prostate, kidney and other body parts during surgery, allowing an alternative first step of spectral histopathological examination and disease state characterization. This technique can open another branch of clinical diagnostics concerned with minimally invasive, fast, remote analysis for endoscopic and catheter applications as well as for the needle regime. Using these techniques, a high sensitivity for the composition of body fluids such as blood, saliva, urine, lymph and gland system is achieved as well.

This invention relates primarily to diagnostics of normal and pathological human skin tissue in vivo, where the sensor probe has direct contact with the patients skin tissue. As an example of this approach, we can distinguish and diagnose healthy, tumorous, precancerous and cancerous tissue of the skin on a molecular level in specific IR spectral ranges (fingerprint regions).

The invention provides a powerful method to detect functional molecular groups to elucidate complex structures within tissue, to characterize, distinguish and diagnose healthy, tumorous, precancerous and cancerous tissue at an early stage of development. More particularly, the invention provides important information such as the absorbance measured as a peak position, peak height, peak height ratio, peak area or peak area ratio from the obtained FTIR tissue spectra.

In a broad sense, the invention is also directed to a new method and compact apparatus with several fiber optical probes and accessories for obtaining response data by examining biological tissue under the influence of the environment, for example sun-induced aging of the human skin or treatment for aging skin and diagnostics of acupuncture points and normal human skin zones.

### SUMMARY OF THE INVENTION

The subject of the present invention is noninvasive tissue diagnostics in vivo using a combination of FTIR spectroscopy method with fiber optical techniques. In accordance with the present invention unclad optical fibers and fiber probes in the regime of ATR are applied to living tissue of animals and humans. A beam of infrared radiation (preferably middle infrared radiation) is passed through a low loss optical fiber and interacts with the tissue via the ATR effect. In this process, the absorbing tissue is placed in direct contact with the reflecting fiber.

The length of interaction of the tissue surface with a cylindrical flexible fiber probe varies from about 1 to 10 mm. The depth of penetration of the infrared light in living tissue is of the order of the wavelength used. Silver halide fibers are characterized by an index of refraction n₁ of approximately 2.2 whereas living tissue has an index of refraction close to water with n₂=1.3. Therefore the ATR condition n₁>n₂ is satisfied and the multiply reflected wave can be detected and analyzed by a FT spectrometer. In the case of very small biopsy samples the flexible fiber probe can be bent at specific angles. In addition, infrared needle probes of the present invention can be used for fluid and tissue diagnostics, in particular for minimally invasive biopsy techniques. Furthermore this invention includes compact fiber optic probes for endoscopic and/or catheter applications. For example the needle probes are also suitable for investigations of breast cancer and prostate cancer. Moreover, this regime of minimal invasive biopsies has a great potential for body fluid analysis.

The optical fiber elements for ATR probes are commonly polycrystalline AgBrₓCl₁₋ₓ (where x=0 to 1) fibers, typically 1 mm in diameter. They operate in the spectral range 3 to 20 μm with low optical losses, typically 0.1 to 0.5 dB/m at 10 μm. A preferred fiber probe is characterized by a high flexibility (R_{bending}>10 to 100 fiber diameters) depending on the concentration of bromine and chlorine, structure, purity of composition and manufacturing process. These type of infrared fibers are soft, nontoxic and nonhygroscopic. The optical system consists of the optical fibers to input and output the infrared radiation and focusing spherical mirrors or lenses to focus an infrared beam into the fiber and collect light from the fiber onto a cooled detector (preferably a nitrogen cooled MCT detector). The optical scheme of the invention is specifically designed and applicable with any commercial FT spectrometer.

The fiberoptic evanescent wave Fourier transform (FEW-FTIR) spectra measured in vivo enable the user to select specific spectral ranges, where fundamental changes in the protein, lipid, phosphate, and sugar systems as well as hydrogen bonds occur. Such FEW-FTIR spectra reveal important information about “order-disorder” phenomena in living tissue and hence the disease state.

A preferred embodiment involves ATR fiber optical probes for fast, remote (up to 3 m), noninvasive and nontoxic diagnostics of skin cancer in vivo and ex vivo during surgery and following incisions.

Another preferred embodiment is the measurement and disease state characterization of human skin tissue in vivo in the spectral range from 800 to 3700 cm⁻¹. Specifically the spectral variation from normal to pathological tissues is indicated in the regions of 800 to 1500, 1500-1800, 2700-3100, and 3100 to 3700 cm⁻¹. The group of bands between 800 and 1500 originate mainly from molecular vibrations of sugars, phosphate groups, and amide III. The spectra obtained in the 1500 to 1800 cm⁻¹ wavenumber region stem from amide I, amide II, and two resolved carbonyl bands. The range from 2700 to 3100 cm⁻¹ is dominated by C—H symmetric and asymmetric stretching vibrations. Bands arising from amide A (O—H and N—H vibrations) occur in spectral region from 3100 to 3700 cm⁻¹ (Anthony R. Rees and Michael J. E. Steinberg, From Cells to Atoms, Blackwell Scientific Publications, Oxford (1994)).

A further preferred embodiment is the analysis and means for analyzing the pronounced variation of these specific bands from normal, precancerous, to cancerous skin tissue measured in vivo. In particular this diagnostic method is very sensitive to diagnose early stages of skin cancer and precancerous phenomena. Benign and non-benign tumors can be clearly differentiated by the FTIR method. This type of skin diagnostics is ideal for surface investigations because the depth of IR light penetration is about 10 to 20 μm depending on the wavelength. The method can also be applied to skin aging involving changes of both intrinsic aging and sun-induced aging (photoaging or dermatoheliosis).

Another preferred embodiment is the diagnostics of normal skin tissue, including the surface response from different acupuncture points and skin zones of the human body. This method is a more selective technique on a molecular level when compared to traditional acupuncture diagnostics, such as electroacupuncture.

In summary the FEW-FTIR spectroscopy technique using fiber optical sensors provides a new effective, fast method for characterization of normal, cancerous, and otherwise diseased skin tissue. The changes in tumor spectra can be observed in real time and analyzed by state of the art pattern recognition and neural network computer programs. Finally the method is very sensitive to the influence of the environment on skin tissue damage. Another advantage of this method is potential applications to any environment related health problems.

### BRIEF DESCRIPTION OF THE DRAWINGS

In the drawings:

FIG. 1 is a schematic illustration of a preferred embodiment of the diagnostics method of the present invention

FIG. 2 is a block diagram showing the principle of tissue diagnostics using the present invention

FIGS. 3a, b and c are schematic views of different middle infrared (MIR) fiber probe embodiments of the present invention

FIG. 3d is a schematic view of an endoscope or catheter embodiment of the present invention

FIG. 4 is a remote FEW-FTIR spectrum of normal skin measured in vivo. The measurement time is about 40 seconds.

FIGS. 5a to 5d show typical FEW-FTIR spectra of normal human skin tissue in vivo in the practice of this invention. The dotted lines represent computer fits of the main observed band structures. Lorentzian profiles have been used as fitting functions. The spectra originated from remote measurements.

FIGS. 6a, b, and c are schematic diagrams of methods for normal and cancer tissue diagnosis in accordance with the invention.

a.) in vivo

b.) ex vivo

c.) incision (under epidermis)

FIG. 7 shows several in vivo FEW-FTIR spectra of “normal” human skin close to a benign tumor produced by the method of the present invention.

FIG. 8 shows several in vivo FEW-FTIR spectra of a pigment nevus (noncancerous) in vivo for several patients, produced by the method of the present invention.

FIG. 9a displays in vivo measurements of FEW-FTIR spectra of normal (A) and malignant (B) skin tissues (premelanoma case) in the range of 1480-1850 cm⁻¹. The spectra were recorded using the method of the present invention.

FIG. 9b shows ex vivo measurements of FEW-FTIR spectra of normal (A) and malignant (B) skin tissues (premelanoma case) in the range of 1480-1850 cm⁻¹. The spectra were recorded using the method of the present invention.

FIG. 10a indicates in vivo measurements of FEW-FTIR spectra of normal (A) and malignant (B) skin tissues (melanoma case) in the range of 1480-1850 cm⁻¹. The spectra were recorded using the method of the present invention.

FIG. 10b represents ex vivo measurements of FEW-FTIR spectra of normal (A) and malignant (B) skin tissues (melanoma case) in the range of 1480-1850 cm⁻¹. The spectra were recorded using the method of the present invention.

FIG. 11 shows in vivo measurements of FEW-FTIR spectra of normal (A) and malignant (B) skin tissues (basaloma case) in the range of 1480-1850 cm⁻¹. The spectra were recorded using the method of the present invention.

FIG. 12a shows in vivo measurements of FEW-FTIR spectra of normal human skin in the range of 850-1800 cm⁻¹ for three different body locations, namely the left elbow crease (LU5), lower lip and left ear. The spectra were recorded using the method of the present invention.

FIG. 12b indicates in vivo measurement of FEW-FTIR spectra of normal human skin in the range of 2450-4000 cm⁻¹ for three different body locations, namely the left elbow crease (LU5), lower lip and left ear. The spectra were recorded using the method of the present invention.

FIG. 13a shows in vivo measurements of FEW-FTIR spectra of normal human skin in the range of 850-1800 cm⁻¹ for two acupuncture points of the left wrist. The spectra were recorded using the method of the present invention.

FIG. 13b represents in vivo measurements of FEW-FTIR spectra of normal human skin in the range of 2450-4200 cm⁻¹ for two acupuncture points of the left wrist. The spectra were recorded using the method of the present invention.

FIGS. 14a-e indicate in vivo measurements of FEW-FTIR spectra of normal skin in the range of 1500-1800 cm⁻¹ for five different acupuncture points: a)lower lip, b) left ear, c) elbow crease (LU5), d) left wrist (8p), and e) lower wrist (9p). The spectra were recorded using the method of the present invention.

FIGS. 15a-e show in vivo measurements of FEW-FTIR spectra of normal skin in the range of 2800-3000 cm⁻¹ for five different acupuncture points: a)lower lip, b) left ear, c) elbow crease (LU5), d) left wrist (8p), and e) lower wrist (9p). The spectra were recorded using the method of the present invention.

### DETAILED DESCRIPTION OF THE INVENTION

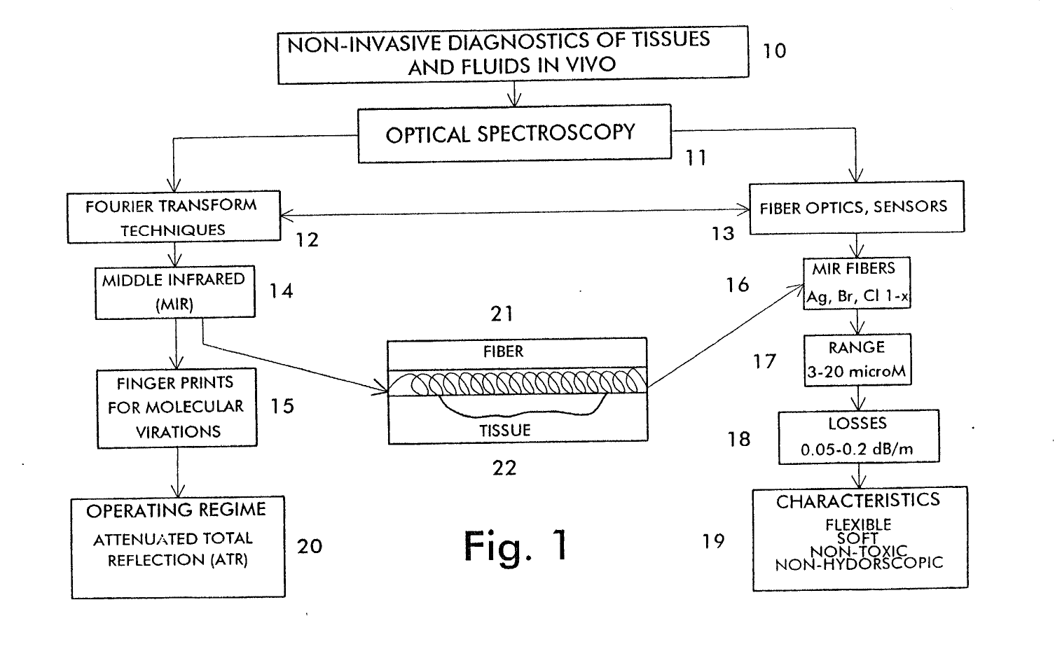

With reference to the diagrammatic view of FIG. 1, illustrating noninvasive diagnostics of tissue and fluids in vivo 10, this method is connected to the field of optical spectroscopy 11, and in particular to Fourier Transform techniques 12 in combination with fiber optics and sensors 13. Tissue measurements are performed in the middle infrared (MIR) 14, and recorded spectra are fingerprints for specific molecular vibrations 15. Specialized MIR fibers of the type AgBrₓCl₁₋ₓ 16, operated in the range 3-20 μm with a diameter D<=1 mm 17 and extremely low losses 18 have unique properties such as high flexibility and softness, and are nontoxic and nonhygroscopic 19. The unclad MIR fibers 16 are designed for the attenuated total reflection (ATR) regime 20. Fiber probe 21 is in direct contact with the tissue 22.

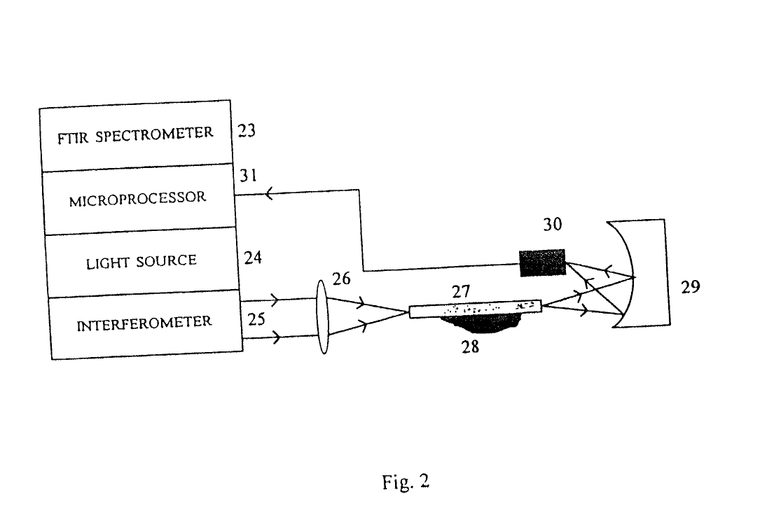

The general nature and usage of the apparatus in accordance with the invention is illustrated in FIG. 2. The optical scheme consists of a commercial FTIR spectrometer 23. Light from an IR source 24 passes through a Michelson interferometer setup 25, and is for example extracted through an external port and focused into an unclad optical fiber. The optical scheme of this invention consists of optical fibers and fiber probe 27 to input and output the infrared radiation via focusing lenses or spherical mirrors 26,29. In accordance with the invention the unclad fiber probe is in direct contact with the tissue sample 28, where the length of contact between the fiber and tissue varies from one to a few millimeters. In accordance with this invention the unclad fiber has direct contact with the tissue similar to the prism in the ATR method.

At the tissue-fiber interface, an evanescent wave penetrates beyond the tissue surface into the sample. An evanescent wave is characterized by a nonpropagating field in the optically denser medium, whose electric field amplitude decays exponentially with distance from the surface. The reflected light is collected from the tissue-fiber interface onto a detector, preferably a nitrogen-cooled MCT (Mercury, Cadmium, Tellurium) detector 30. After amplification the signal is processed in a microprocessor or computer system 31. It is further noted that a larger tissue-fiber contact corresponds to a more pronounced FTIR tissue spectrum. Depending on the signal to noise ratio an optimal number of scans can be chosen for in vivo tissue measurements. Typical recording times range approximately from 2 to 40 seconds. Therefore this diagnostic technique is very convenient for human patient and animal testing.

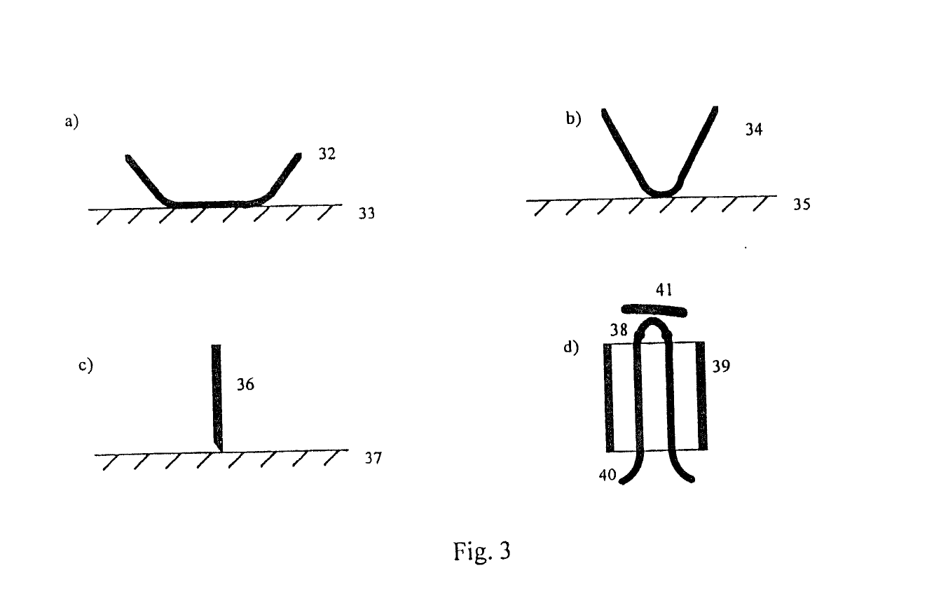

A schematic view of different fiber probes in close contact with the tissue are depicted in FIGS. 3a to 3d. An embodiment of these probes is that the fibers, preferably silver halide fibers, can be bent to a specific form and angle creating different tip probes depending on the size of the tissue samples. The probes of this invention can be utilized with different radii of curvature of the tip portion. In FIG. 3a is shown an unclad MIR fiber tip probe 32 covering a larger tissue segment 33. Another exemplary utilization of the tip probe is indicated in FIG. 3b. Here the MIR fiber 34 is bent at a sharp angle, forming a tip probe for detection of smaller areas of tissue 35. This probe is suitable for detection of normal and malignant tissues with size of the order of 1 mm or less. Such small tip probes, typically 1 mm in diameter, can also be used for biopsies. Another embodiment of the probe is shown in FIG. 3c, wherein a needle tip 36 touches a tissue surface 37. This probe of the present invention is used in minimally invasive diagnostics, for example for breast cancer. The same probe can be used as well in measurements of fluids. A further embodiment of our invention of the probe 38 touching the tissue 41 is shown in FIG. 3d, wherein an endoscope or catheter 39 is illustrated with additional remote fiber cable 40.

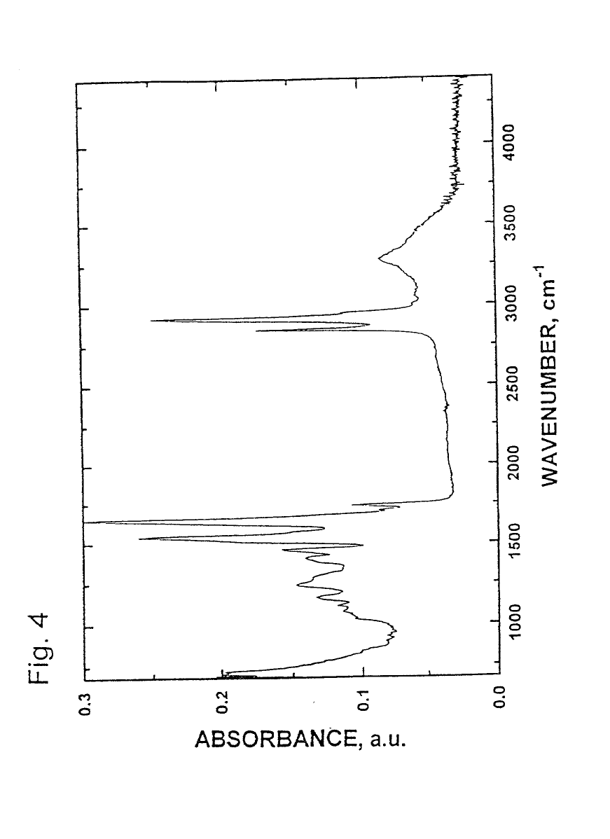

This type of sensor in accordance with the invention can be applied for breast, kidney, stomach, lung, and prostate cancer diagnostics. The fiber probes shown in FIGS. 3a to d are easily changed and are generally used only one time. For fluid examination the fiber probe is located within the hypodermic needle or syringe. In this invention changeable tip probes are used for biopsy and endoscopic applications. The special tip size and configuration allow the collection or scattering of IR light for different type of tissue examinations. In another embodiment (see FIG. 4) a typical remote FEW-FTIR spectrum of normal skin in vivo in the range of about 500 to 4500 cm⁻¹ is displayed. In this spectrum the absorbance is plotted versus the wavenumber in cm⁻¹ and the spectrum is measured with a resolution of 4 cm⁻¹.

Polycrystalline silver halide AgBrₓCl₁₋ₓ fibers, preferably with 1 mm diameter, extremely low optical losses (0.1 to 0.5 dB/m in the region of 10 μm), and high flexibility (R_{bending}>10 to 100 fiber diameters) are used as fiber tip probes (Artjushenko et al. , U.S. Pat. No. 5,309,543 and U.S. Pat. No. 5,342,022, and Küpper and Butvina, Offenlegungschrift DE 4414552A1). As can be seen from FIG. 4, the fiber probes transmit IR radiation with low losses in the range of about 800 to 4000 cm⁻¹ Hence, in accordance with one aspect of the invention the quality of the obtained IR spectra is high, i.e. low background, excellent statistics and fill compensation in the region of water vapor and CO₂ vibrations.

Another embodiment of the human skin diagnostic in vivo is related to different fingerprint regions of the IR spectra in the wavenumber ranges 800 to 1500 cm⁻¹, 1500 to 1800 cm⁻¹, 2700 to 3100 cm⁻¹, and 3100 to 3700 cm⁻¹. In the present invention, the FEW-FTIR method of tissue diagnostics in the above ranges of spectral measurements can be extended to the near infrared (NIR) of far infrared (FIR) regions using different fiber materials and fiber probes.

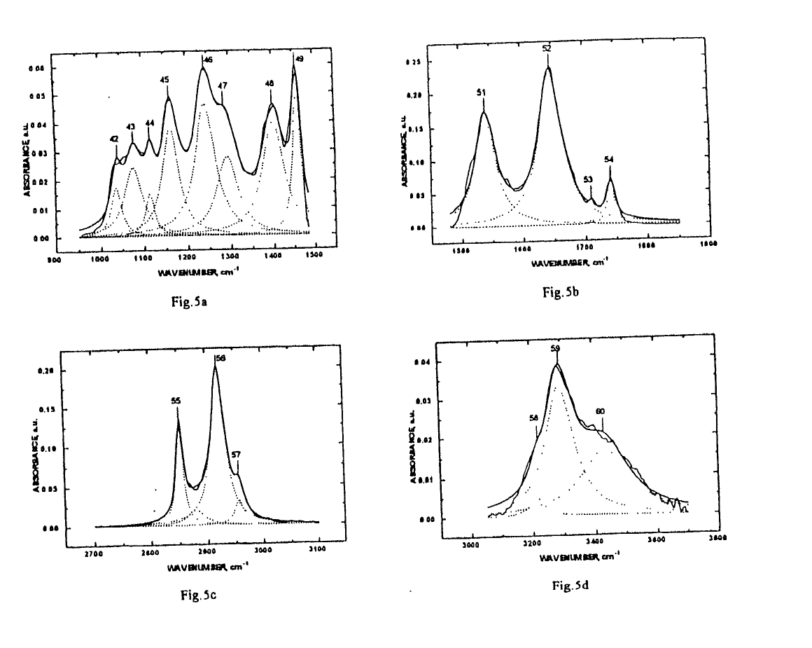

The present invention is further embodied in the in vivo FEW-FTIR spectral features of normal human skin tissue shown in FIGS. 5a to 5d. FIG. 5a indicates the significant IR bands of 42 to 49 connected with vibrations in systems of phosphate groups, sugars, amide III and CH₂ deformations. In particular, in accordance with the invention peaks 42 and 43 belong to vibrations of the C—O—C groups in sugars. Peak 44 is attributed to symmetric stretching modes of phosphate groups (PO₂⁻). Furthermore peak 45 coincides with stretching vibrations of C—O and C—C bands in systems of sugars. The structure labeled 46 originates from asymmetric stretching of phosphate groups (PO₂⁻) plus associated C—O—C bands in sulfoglycolipids, whereas peak 47 stems from amide III band components of proteins. Peak 48 of this invention is due to symmetric stretching of carboxylate groups (COO⁻) and finally peak 49 corresponds to the bending of methylene (CH₂). All of these band structures can be used as fingerprints for tissue diagnostics, and are related to this invention.

As may be seen in FIG. 5b, four main bands contribute to the FEW-FTIR spectrum of normal skin tissue in the range of the dominant amide bands. Thus peak 51 is associated with amide II vibration and peak 52 is due to amide I of a helical structure for normal skin. In addition two weaker bands, 53 and 54, are assigned to C═O aliphatic and C═O cyclic groups, respectively. In accordance with the present invention FIG. 5c shows three major band structures, 55 56 and 57. Bands 55 and 56 correspond to symmetric and asymmetric stretching of methylene group (CH₂) in systems of fatty acids, and shoulder 57 of the band 56 is due to asymmetric stretching of methyl group (CH₃). All of these bands play an important role in tissue diagnostics and are therefore an embodiment of this invention.

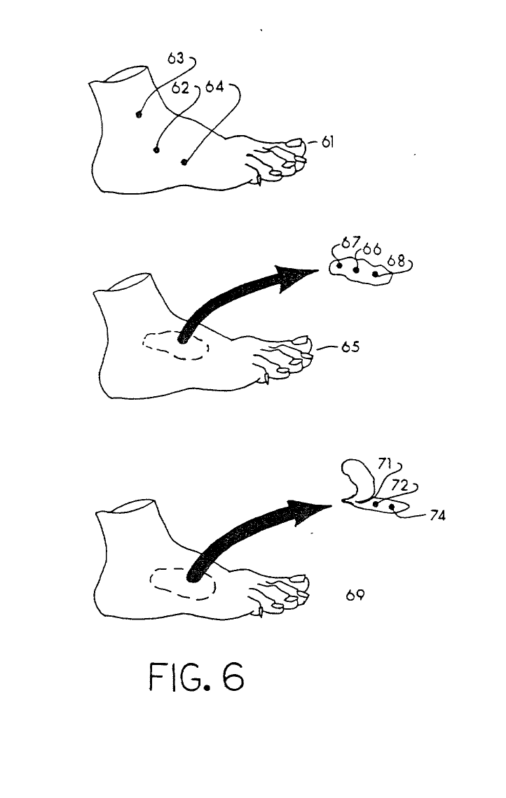

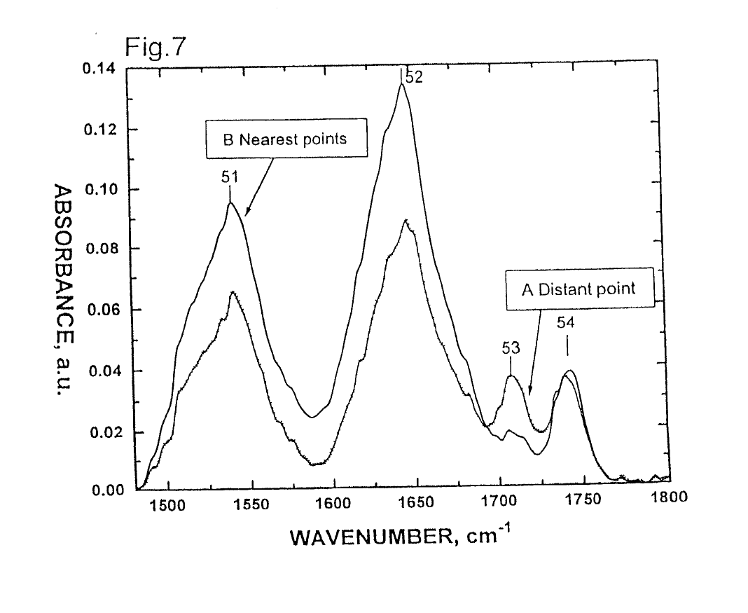

Another embodiment of our invention is associated with the FEW-FTIR spectrum of normal skin tissue in the range of about 3100 to 3700 cm⁻¹. The band structure labeled 59 with shoulder 58 belong to NH stretching modes in the amide A system of proteins, and the partially resolved band 60 originates from OH stretching. The same FTIR-FEW approach can be applied to tumor diagnostics and disease state characterization of skin tissue. Therefore this invention relates also to cancer diagnostics in early and advanced stages. FIGS. 6a, b,and c depict clinical procedures for analyzing skin tissue material in vivo and ex vivo during surgery, and in incisions (in vitro).

FIG. 6a indicates a sequence of measurements of human skin 61 in vivo (directly on patient), where point 62 is the center of tumor or cancer and the points 63 and 64 correspond to measurements taken in the direction of normal skin. The distance between 62-63 and 62-64 depend on the size and growth of the tumor tissue. FIG. 6b shows the scheme of exvivo measurements at the surface off skin tissue 65 after surgery. Here 66, 67 and 68 correspond to the same locations (62, 63 and 64) indicated in FIG. 6a. Moreover FIG. 6c shows a characteristic cut 69 at the center of a tumor 70 and distant points 71 and 72 to measure different layers of the tumor and normal skin below the skin surface. Such experiments can be performed conveniently in any surgical center (operating room) for ex vivo examinations during surgery. This method applies to breast cancer and tumorous tissues from lung, kidney, prostate, stomach, glands etc. for on-line, remote, fast, nondestructive diagnostics. The results of such spectral measurements can be compared directly with the traditional and more time consuming analysis of histological data. This new IR spectral histology method in vitro is in accordance with the present invention.

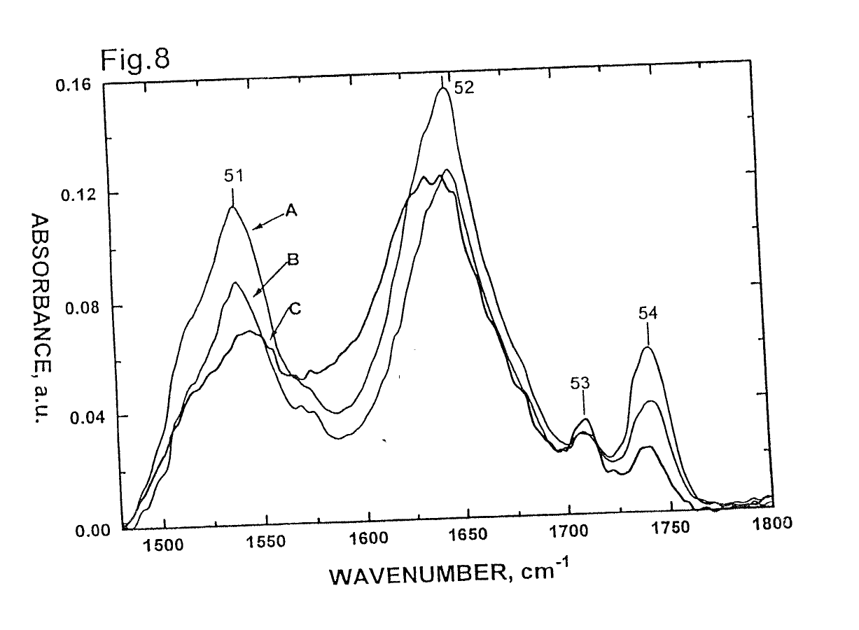

FIG. 7 demonstrates the sensitivity of FEW-FTIR non invasive measurements of skin tissue in vivo. For example FTIR spectra of normal skin (A), distant point (see FIG. 6a, point 63) exhibit four distinct bands in the range of the main amide vibrations (see FIG. 5b). In contrary the spectrum of nearest point (B) to tumor (see FIG. 6a, point 64) shows only three distinct bands, where the structure labeled 53 (see FIG. 5b) is reduced and nearly disappears in curve (B). Furthermore FIG. 8 indicates a typical FEW-FTIR spectra arising from pigment nevus (noncancerous) for three different patients (A,B,C). It is evident that in two cases (A and B) the four band positions 51 to 54 coincide, but in the case C the peak positions 51 and 52 originating from amide I and amide II are shifted. This is a clear indication of an early stage of cancer revealed by an apparatus according to the present invention.

The invention is also concerned with means for comparing band structure, peak positions, peak ratios etc., including visual displays of the spectra to be compared. Alternatively, such means for comparing can be superimposed. It is also possible to provide more sophisticated means for comparing which calculate differences between different spectra, e.g. subtracting one spectrum from another spectrum in order to reveal the differences between the spectra.

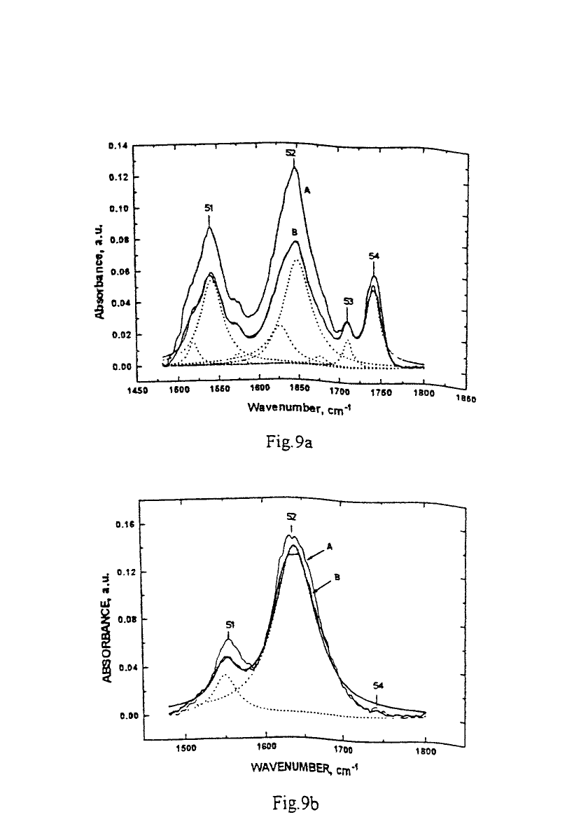

Accordingly another object of this invention is to provide a method and means for the diagnostics of premelanoma in vivo as shown in FIGS. 9a and b. When comparing normal (A) and premelanoma (B) tissues (see FIG. 9a), we find that the four main band structures and the mean peak positions have not changed, whereas the relative intensities of both amide bands decreased. A practicable, reliable method available in this invention for monitoring cancer and precancer is the determination of intensity ratios for three band pairs: R_{I}(I₅₂/I₅₁), R_{II}(I₅₂/I₅₄), and R_{III}(I₅₄/I₅₃). In particular the intensity ratio R_{II} can be used for cancer and precancer diagnostics. In FIG. 9b is shown a comparison of FEW-FTIR ex vivo measurement (incision) for normal (A) and malignant (B) skin tissue (premelanoma) in the same range as in FIG. 9a. From this figure it is apparent that the two hydrogen bonded carbonyl bands 53 and 54 disappeared completely in spectra of incision under the top layer of epidermis. In addition the intensity ratio R_{I} has changed substantially and the peak positions of the bands 51 and 52 have shifted in opposite directions.

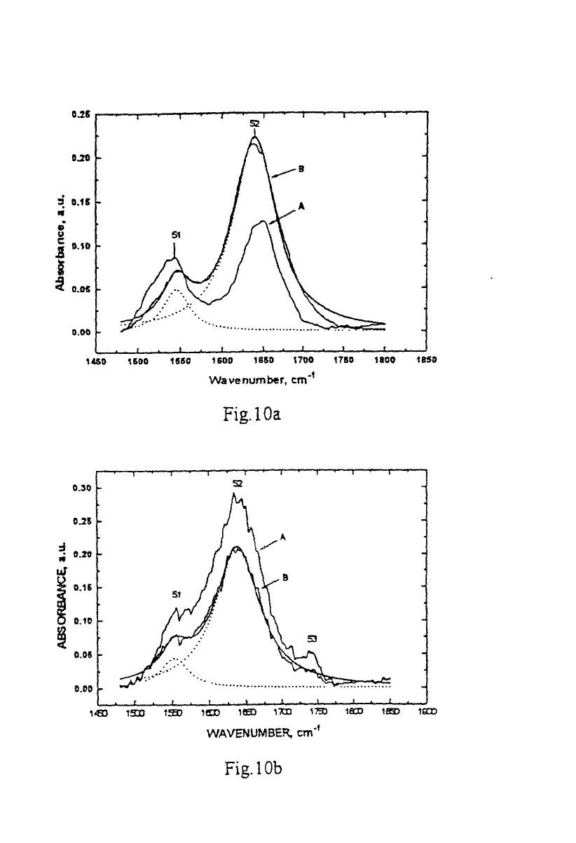

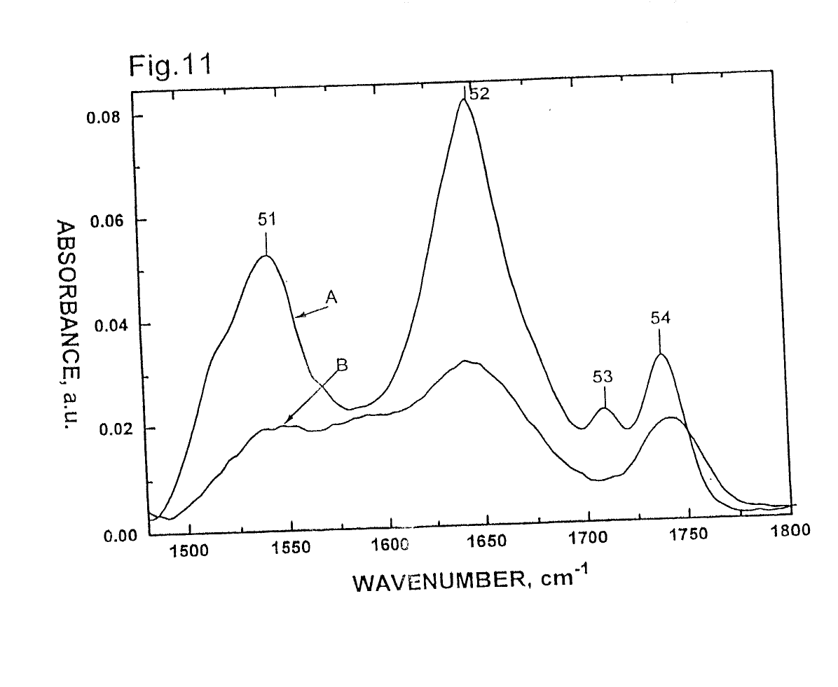

As another example of the foregoing diagnostic technique we display in FIGS. 10a and b an extreme case of melanoma. As can be seen from FIG. 10a both carbonyl bands 53 and 54 are absent for normal (A) and malignant (B) skin surface points (see FIG. 6a). Furthermore the band maxima 51 and 52 exhibit characteristic shifts. Hence the distances in band position between 51 and 52 can be used as another parameter for cancer diagnostics. In addition there exists a pronounced difference in the intensity ratio for R_{I} in accordance of this invention. As can be seen in FIG. 10b, dramatic changes occur in the FEW-FTIR spectra from normal (A) and malignant (B) skin tissue (melanoma) below the epidermis (see FIG. 6c) in the same range compared to FIG. 10a. It is further noted that the peak 51 has partially collapsed. However a weak contribution of band 54 (carbonyl group) is observed exclusively for normal tissue.

With the apparatus of this invention FEW-FTIR spectra of malignant skin tissues in vivo (basaloma) have been measured as indicated in FIG. 11. In this figure are displayed spectra for normal (A) and malignant (B) skin surfaces. Significant differences occur in peak positions, intensities, intensity ratios and shape of band structures. Therefore basaloma can be detected directly from the skin surface by comparing curve A and B (see FIG. 11). Furthermore melanoma can be analyzed at the surface and below the surface of the skin.

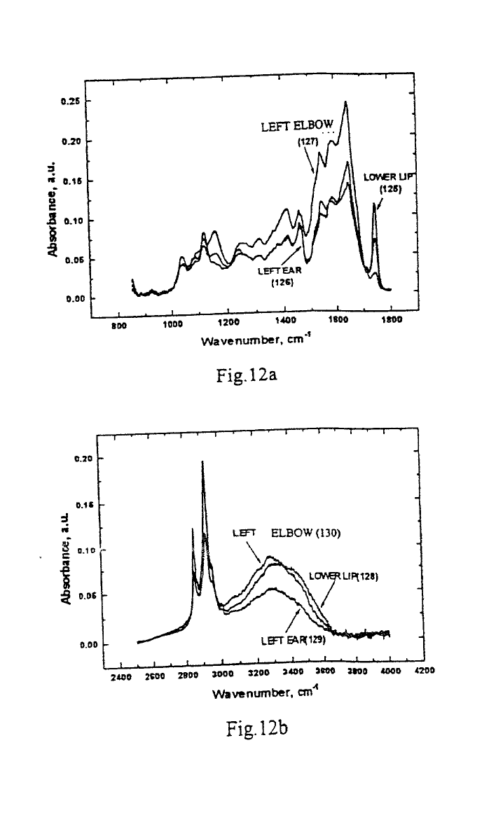

Another embodiment of this invention is an apparatus and method for noninvasive, fast, direct, sensitive investigations in vivo of various human skin points and zones including acupuncture (AC) points in the range of about 800 to 4000 cm⁻¹. Acupuncture is an ancient Chinese diagnostic and treatment method (Ralph Alan Dale, Demythologizing Acupuncture, Alternative Complementary Therapies (1997)) in which electrodes or needles are used at specific points, connected with specific organs. These acupuncture points are characterized by comparitively low electrical resistance, and are well mapped. The subject of this invention includes the surface response of different acupuncture points of the human body using the FEW-FTIR method of this invention, for the purposes of disease state characterization and development of new acupuncture techniques. FIGS. 12a and b represent IR spectra showing an extremely sensitive surface response of several AC points and differences between various AC points, for example between lower lip 125 (RN24, middle of the mentolobial groove) (Wu Shao, Body Model for Both Meridian and Extraordinary Points of China, GB 123 46-90), left ear 126, left elbow crease 127 (LU5 elbow crease) in the spectral range of 800 to 1800 cm⁻¹. In FIG. 12b are shown spectra associated with the same points in the spectral interval 2500 to 4000 cm⁻¹. In accordance with this invention and the apparatus provided by the invention the peak positions, intensities, widths, shapes, and intensity ratios of bands can be compared. In particular the amide I and II region is sensitive to Watson-Crick pairing. For example the appearance of the 1585 cm⁻¹ structure, appearing in the spectra of the lower lip 125, left ear 126, and left elbow crease 127 represents C═O stretching modes in guanine. Another important fingerprint region of human skin AC points detected in the range 2500 to 4000 cm⁻¹ (see FIG. 12b) is concerned with C—H, N—H, and O—H vibrations, as demonstrated for lower lip 128, left ear 129 and left arm 130. It can be seen that pronounced differences among the different spectra are obvious in the system of amide A (proteins) connected with N—H and O—H groups and lipid groups connected with C—H vibrations.

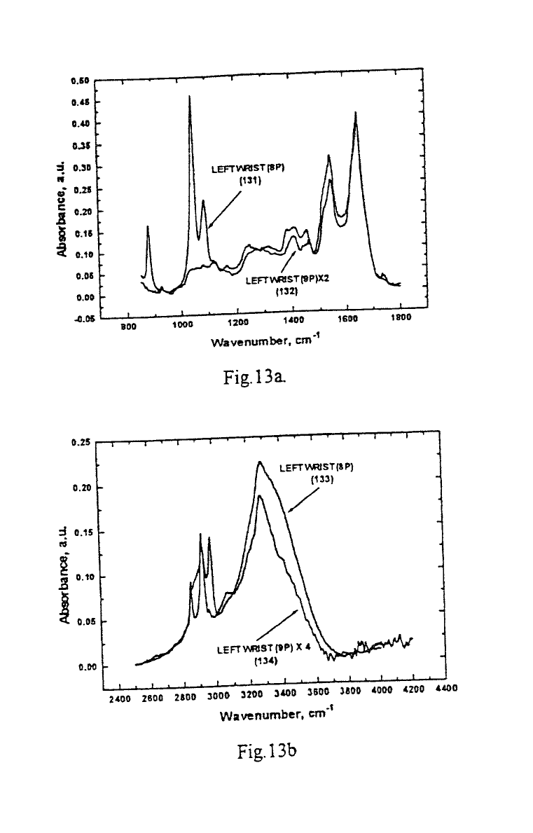

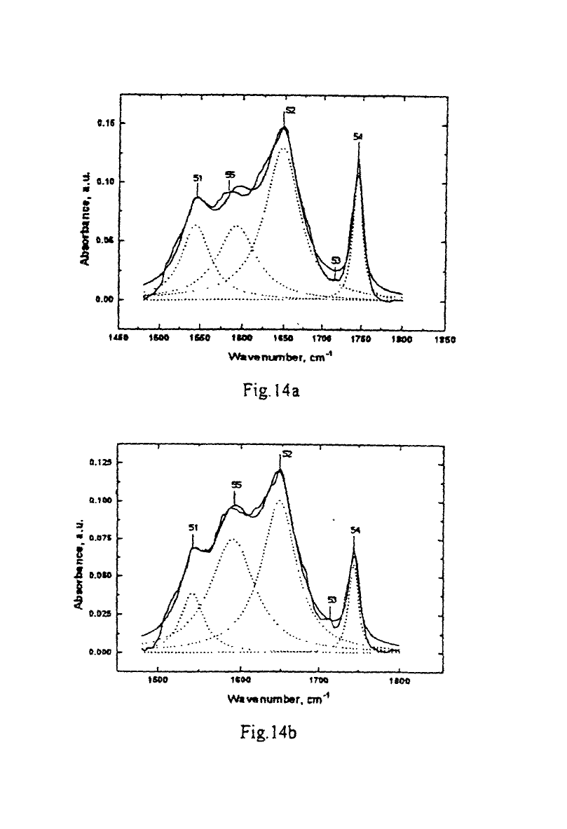

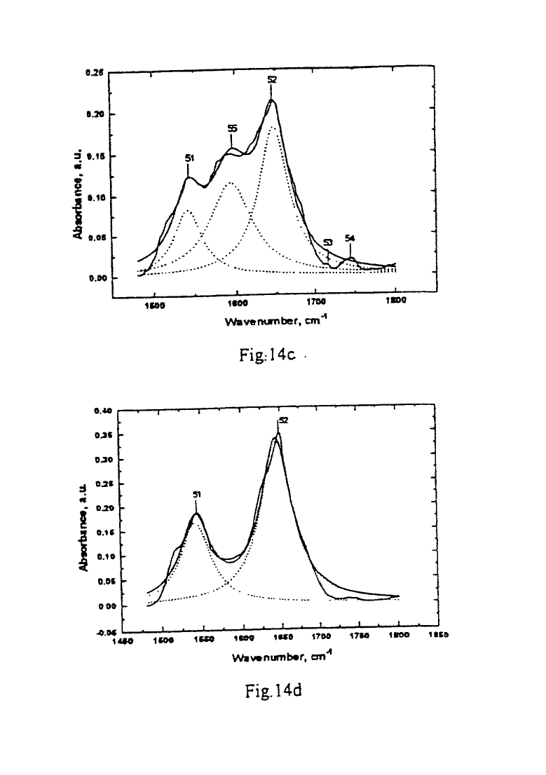

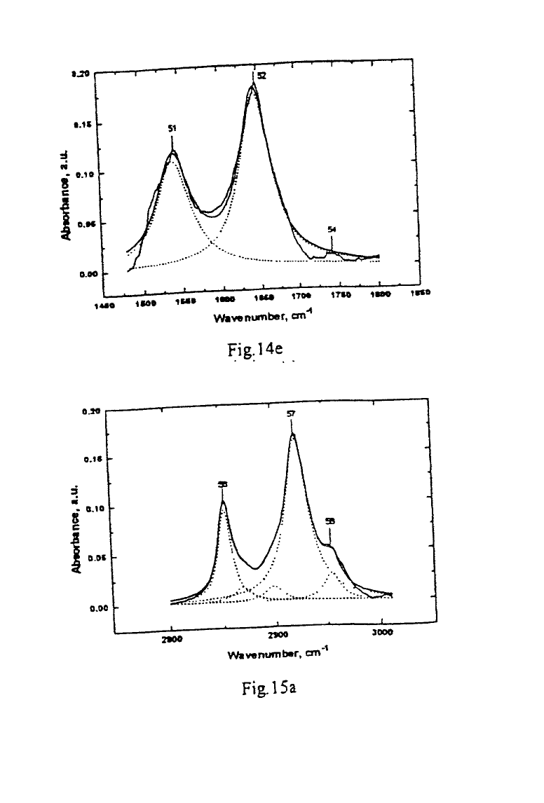

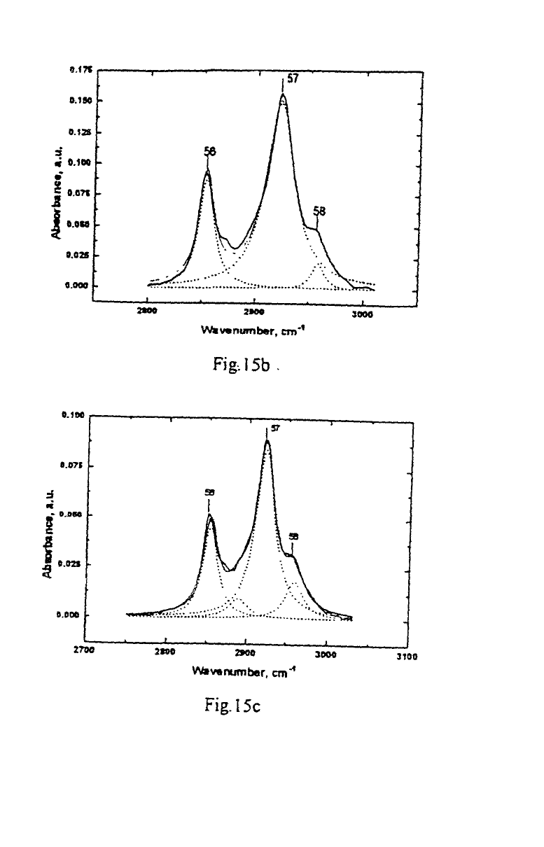

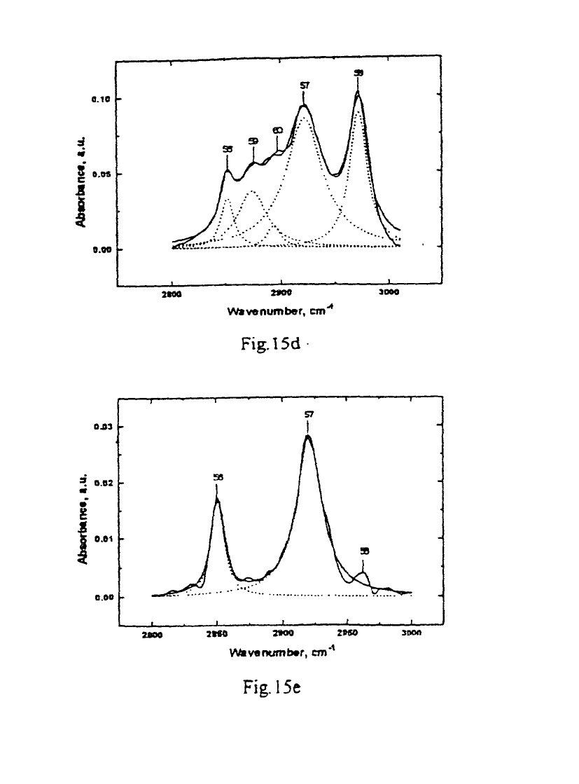

FIGS. 13a and 13b show results for two AC points on the wrist, namely LU8 (8P) and LU9 (9P). In particular in FIG. 13a are indicated the IR spectra results (800 to 1800 cm⁻¹) for LU8,131 and LU9,132. Huge differences are observed in the spectral range 800 to 1200 cm⁻¹ attributed to phosphate groups in lipid systems of human tissue. The higher wavenumber range for the same AC points LU8 (8P) 133 and LU9 (9P) 134 is illustrated in FIG. 13b, where the C—H vibrations due to aliphatic chains in lipids show large differences. In the following detailed spectra (FIGS. 14a to e), showing a spectral deconvolution of the main amide bands (1450-1800 cm⁻¹) in the MIR range. In FIGS. 15a to e the same AC points are represented in another spectral interval of C—H vibrations in the region of 2800-3000 cm⁻¹.

The bands 51,52,and 54 are assigned to vibrations of hydrogen bonded amide II, amide I and carbonyl groups. In the three cases of lip, ear, and elbow crease an additional band at 1590 cm⁻¹ (55) is apparent (FIGS. 14a-c) connected to Watson-Crick base pairing. In FIGS. 14d and e this band, as well as the carbonyl bands (54), are absent. These differences are connected with the content of lipids and/or proteins in tissue. The present invention is embodied in the appearance and disappearance of the band structures 53,54, and 55 as well as in the intensity ratio I(52)/I(51) corresponding to the amide I and amide II bands. Another object of the present invention is concerned with the bands 56,57,58,59, and 60 in the wavenumber range 2800 to 3000 cm⁻¹ (see FIGS. 15a to e). In all cases displayed in FIGS. 15a to 15e peak 56 is assigned due to C—H symmetric stretching in methylene groups (CH₂) of lipids. The band structure located at about 2922 cm⁻¹ is identified as the asymmetric stretching of methylene groups CH₂ in lipids. Furthermore peak 58 at approximately 2956 cm⁻¹ arises from asymmetric stretching vibration of methyl group (CH₃). When comparing the spectra in FIGS. 15a,b,c,and e, the spectrum associated with the left wrist, acupuncture point Lu9 (9P) differs in the weak intensity of the band 58 (see FIG. 15c). This change depends on the vibration of the methyl group. A special situation arises for the spectrum from FIG. 15d (AC point 8P or LU8). Here peak 58 is dominating the spectrum. In addition two new band features near 2874 cm⁻¹ (59) and 2893 cm⁻¹ (60) are observed originating from symmetric stretching vibration of methyl group (CH₃) and C—H stretch.

It can be seen that the pronounced peak 58 occurring at 2972 cm⁻¹ is shifted substantially towards higher wavenumbers (Δv˜16 cm⁻¹) when compared to the band structures 58 shown in FIGS. 15a,b,c and e. Therefore, peaks 58,59,and 60 can be used as fingerprints for AC diagnostics.

In conclusion the infrared FEW-FTIR spectroscopic technology described in this invention is not only very sensitive to cancer and precancer diagnostics of human tissue, but also for the diagnostics of normal skin and even for the characterization of specific acupuncture points. In particular this invention relates to the surface response of human tissue including AC points.

It is understood that the invention is not confined exclusively to the particular embodiments on human skin described herein as illustrative, but embraces the disease state characterization of other forms thereof within the scope of the following claims.

53. A system for analysis of biological samples, comprising:
(a) a middle infrared radiation source configured to provide radiation in a spectral range of between approximately two and one half microns and approximately twenty microns;
(b) an optical fiber, operatively coupled to said middle infrared radiation source, said optical fiber being substantially transparent in said spectral range of between approximately two and one half microns and approximately twenty microns;
(c) an interchangeable fiberoptic probe associated with said optical fiber and configured to direct radiation from said radiation source to said biological sample;
(d) a detector operatively coupled to said optical fiber and configured to detect radiation reflected from said biological sample through said optical fiber; and
(d) a Fourier transform infrared spectrophotometer operatively coupled to said detector and configured to detect radiation in said spectral range of between approximately two and one half microns and approximately twenty microns.

54. The system of claim 53, wherein said interchangeable fiberoptic probe is selected from a shaped probe, a needle probe, a diffusor probe, a microscope head probe, an endoscopic probe, or a catheter probe.

55. The system of claim 53, wherein said fiberoptic probe is configured for use as an in vivo percutaneceous probe.

56. The system of claim 53, wherein said radiation has a spectral range of between approximately two point five microns and approximately twelve microns.

57. The system of claim 53, wherein said fiberoptic probe is configured to operate in attenuated total reflectance mode.

58. A method for non-invasive in vivo analysis of biological samples, comprising:
(a) obtaining a first Fourier transform infrared spectrum of a first, normal biological sample using a fiberoptic probe operating in an attenuated total reflection mode;
(b) obtaining a second Fourier transform infrared spectrum of a second, abnormal biological sample using said fiberoptic probe operating in said attenuated total reflection mode; and
(c) comparing at least one selected absorption band in said first Fourier transform infrared spectrum to at least one selected absorption band in said second Fourier transform infrared spectrum.

59. The method of claim 58, wherein said comparing comprises comparing a peak position of said at least one selected absorption band in said first Fourier transform infrared spectrum to a peak position of said at least one selected absorption band in said second Fourier transform infrared spectrum.

60. The method of claim 58, wherein said comparing comprises comparing an area under a peak in said at least one selected absorption band in said first Fourier transform infrared spectrum to an area under a peak in said at least one selected absorption band in said second Fourier transform infrared spectrum.

61. The method of claim 58, wherein said comparing comprises comparing an intensity of a peak associated with said at least one selected absorption band in said first Fourier transform infrared spectrum to an intensity of a peak associated with said at least one selected absorption band in said second Fourier transform infrared spectrum.

62. The method of claim 61, wherein said comparing comprises determining an intensity ratio for said peak associated with said at least one selected absorption band in said first Fourier transform infrared spectrum and said peak associated with said at least one selected absorption band in said second Fourier transform infrared spectrum.
